(19) United States
(12) Patent Application Publication
(10) Pub. No.: US 2018/0168984 A1
(43) Pub. Date: Jun. 21, 2018

(54) EYE MAKEUP COSMETIC COMPOSITION WITH ANTI-CREASING ACTION AND IMPROVED SUSTAINING POWER AND METHOD FOR PREPARING SAME

(71) Applicant: COSON CO., LTD., Seoul (KR)

(72) Inventors: Nu Ri KWON, Gochang-gun (KR); Young Ju KIM, Yongin-si (KR); Wha Young LEE, Yongin-si (KR); Chan Gi LEE, Yongin-si (KR)

(21) Appl. No.: 15/735,517

(22) PCT Filed: Jun. 14, 2016
(86) PCT No.: PCT/KR2016/006299

§ 371 Date: Dec. 11, 2017

(30) Foreign Application Priority Data
Jun. 18, 2015 (KR) 10-2015-0086535

(51) Int. Cl. A61K 8/70; A61Q 1/10; A61K 8/25; A61K 8/19; A61K 8/895; A61K 8/891; A61K 8/92; A61K 8/29

(52) U.S. Cl. CPC A61K 8/70; A61Q 1/10; A61K 8/25; A61K 8/19; A61K 2800/436; A61K 8/891; A61K 8/922; A61K 8/29; A61K 2800/262; A61K 8/895

(57) ABSTRACT

The present invention relates to an eye makeup cosmetic composition with an anti-creasing action and improved sustaining power and a method for preparing the same and, specifically, to an eye makeup cosmetic composition with an anti-increasing action and improved sustaining power and a method for preparing the same, wherein the eye makeup cosmetic composition prevents the generation of creases by containing a fluorine-based derivative, improves the adhesive property to the skin, enhances sustaining power through a thin film coated on the skin, stabilizes the fluorine-based derivative by containing a hydrophobic silica ingredient, maintains a smooth feeling of use and clear color when coated on the skin, and enhances the absorptive power of the fluorine-based derivative by containing the fluorine-based derivative and the hydrophobic silica, and thus can display clear eye makeup while the colors on the skin are not smeared or wiped away for a long period of time.

### CROSS-REFERENCE TO RELATED APPLICATION

This application claims the benefit of Korean application No. 10-2015-0086535, filed on Jun. 18, 2015 with the Korean Intellectual Property Office, the disclosure of which is incorporated herein by reference.

### TECHNICAL FIELD

The present invention relates to an eye makeup cosmetic composition with an anti-creasing action and improved sustaining power and a method for preparing the same, and specifically, to an eye makeup cosmetic composition with an anti-creasing action and improved sustaining power and a method for preparing the same, in which the eye makeup cosmetic composition prevents the generation of creases by containing a fluorine-based derivative, improves the adhesive property to the skin, and enhances sustaining power through a thin film coated on the skin.

In addition, the present invention relates to an eye makeup cosmetic composition with an anti-creasing action and improved sustaining power and a method for preparing the same, in which the cosmetic composition contains a hydrophobic silica ingredient to stabilize the fluorine-based derivative, and maintains the smooth feeling of use and the clear color when coated on the skin.

Further, the present invention relates to an eye makeup cosmetic composition with an anti-creasing action and improved sustaining power and a method for preparing the same, in which the cosmetic composition enhances the adsorptive power of the fluorine-based derivative by containing the fluorine-based derivative and the hydrophobic silica, and thus can display clear eye makeup while the colors on the skin are not smeared or wiped away for a long period of time.

### BACKGROUND ART

Color cosmetics are used for the purpose of making a beauty skin by beautifully changing an appearance. The color cosmetics are classified into a base makeup used for uniformizing a skin color of an entire face and covering defects, and a point makeup for partially enhancing the stereoscopic effect of lips, eyes, nails and so on. The base makeup includes a makeup base, a foundation, a powder, and so on, and the point makeup includes lip cosmetics, eye makeup cosmetics, and so on.

The above eye makeup cosmetics include a mascara, an eyeliner, an eyeshadow, an eyebrow pencil, and so on. The eye makeup cosmetics are applied around the eyes to emphasize an outline of the eyes and to give beauty to the shape of the eyes.

In general, the conditions for the eye makeup cosmetics include that: the cosmetics should have no irritation to the eyes because the cosmetics are applied around the eyes; the cosmetics should have fast drying speed to prevent from being wiped away right after application; the cosmetics should be rarely easily blurred due to sweat, tears and oil; the cosmetics should be uniformly applied without scattering of powder; the cosmetics should have no burden when used; the cosmetics should have low possibility of contamination with microorganisms; and the cosmetics should foe easily removed upon cleansing.

For example, with respect to the related art on the eye makeup cosmetic composition, Korea Registered Patent Publication No. 10-1201953 discloses a water-dispersible and emulsified eye makeup composition and a method of preparing the same, which relates to a water-dispersible eye makeup composition including water, a water-dispersible additive having a dispersion stabilizer and including at least one of an emulsifier and a moisturizer, and a low-order titanium oxide having a particle size of 1 μm to 200 μm, wherein the dispersion stabilizer is any one of xanthan gum and carboxyvinyl polymer, the emulsifier is sorbitan monolate, and the moisturizer is propylene glycol.

The above related art has excellent glossiness, it is unnecessary to add high energy during the manufacturing process due to a simple manufacturing, and it is harmless to a human body.

However, the above related art uses xanthan gum or carboxyvinyl polymer for dispersion stabilization of the water-dispersible formulation, in which the above compound is a dispersion stabilizer (organic thickener) having a long molecular structural formula and a large structure, thus the feeling of use becomes heavy and oily, and stickiness and dirt-lump incur when the content increases. Due to the above problems, it is difficult to apply the above related art to an actual manufacturing process which requires mass production.

With respect to the related art to solve the above-mentioned conventional problems, Korean Registered Patent Publication No. 10-2014-0072548 discloses a makeup cosmetic composition having high glossy and sensation of use. The above related art relates to a makeup cosmetic composition including complex powder, in which a coating layer including one kind of oil selected from the group consisting of dipropylene glycol dibenzoate, C12-C15 alkyl benzoate, phenyl dimethicone, diphenylsiloxy phenyl trimethicone, and combinations thereof is formed on a pigment powder surface.

According to the related art, oil having a light feeling of use is used, such that the adhesion may be improved upon application to the skin, the skin may be brightly displayed, and the smooth and glossy skin may be expressed.

However, because the oil used in the above related art is not stable in dispersibility when mixed with pearl and extender pigment, the pearl and the extender pigment sink by gravity, thus an interface is affected according to surface properties of the pigment. For the same reason, the dispersing power of the pearl is sharply decreased and the pearl sinks in the product. Due to the above problem, the stereoscopic effect is reduced upon eye makeup, and the effects on color expression and gloss cannot be obtained with a viewpoint of the feeling of use, because the pearl sinks in a large amount.

In addition, the eye makeup cosmetic material is creased in the double eyelid upon eye makeup, and the makeup was blurred or wiped away in 1 to 2 hours after completion of the eye makeup, such that the double eyelid line was separated from the eye makeup cosmetic material, thus the part around the eyes seems to be dirty and to have wrinkles.

### DISCLOSURE

### Technical Problem

To solve the above problems, the present invention provides an eye makeup cosmetic composition with an anti-creasing action and improved sustaining power and a method for preparing the same, in which the eye makeup cosmetic composition prevents the generation of creases by containing a fluorine-based derivative, improves the adhesive property to the skin, and enhances sustaining power through a thin film coated on the skin.

In addition, the present invention provides an eye makeup cosmetic composition with an anti-creasing action and improved sustaining power and a method fox preparing the same, in which a functional group including an amide group, a phosphate group or a hydroxyl group is bonded to both ends of the fluorine-based derivative to improve the dispersibility of the cosmetic material and express hydrophilic properties in the functional group, such that the moisturizing property is sustained upon application to the skin.

In addition, the present invention provides an eye makeup cosmetic composition with an anti-creasing action and improved sustaining power and a method for preparing the same, in which the eye makeup cosmetic composition contains a hydrophobic silica ingredient to stabilize the fluorine-based derivative, and maintains the smooth feeling of use and the clear color when coated on the skin.

Further the present invention provides an eye makeup cosmetic composition with an anti-creasing action and improved sustaining power and a method for preparing the same, in which the eye makeup cosmetic composition enhances the adsorptive power of the fluorine-based derivative by containing the fluorine-based derivative and the hydrophobic silica, and thus can display clear eye makeup while the colors on the skin are not smeared or wiped away for a long period of time.

### Technical Solution

The present invention provides an eye makeup cosmetic composition with an anti-creasing action and improved sustaining power, the eye makeup cosmetic composition including a fluorine-based derivative, hydrophobic silica, boron nitride, pearl, extender pigment, vegetable oil, and talc.

In addition, the present invention includes 5% to 64% by weight Of a fluorine-based derivative, 5% to 10% by weight of hydrophobic silica, 10% to 25% by weight of boron nitride, 10% to 20% by weight of pearl, 0.5% to 5% by weight of extender pigment, 0.5% to 5% by weight of vegetable oil, and 10% to 30% by weight of talc.

In addition, the fluorine-based derivative of the present invention may be polyperfluoromethylisopropyl ether.

In addition, according to the present invention, a functional group including an amide group, a phosphate group and a hydroxyl group is further bonded to both ends of the fluorine-based derivative.

In addition, the hydrophobic silica of the present invention may preferably include at least one selected from silica dimethyl silylate, silica dimethicone silylate, silicone acrylate and dimethicone.

Further, the present invention Includes the steps of: feeding a fluorine-based derivative into a mixing vessel to heat and dissolve the fluorine-based derivative at 50° C. to 90° C. (S10); feeding hydrophobic silica to the mixing vessel and agitating the mixture by using an agi mixer to prepare a fluorine-based derivative-hydrophobic silica base (S20); sequentially and slowly feeding boron nitride, pearl, extender pigment, vegetable oil and talc to the base to uniformly mix the mixture by using the agi mixer (S30); and feeding the mixed cosmetic material into a kneader to knead the mixed cosmetic material to prepare a final eye makeup cosmetic composition (S40).

### Advantageous Effects

According to the eye makeup cosmetic composition with the anti-creasing action and the improved sustaining power and the method for preparing the same of the above-mentioned present invention the eye makeup cosmetic composition can prevent the generation of creases by containing a fluorine-based derivative, improve the adhesive property to the skin, and enhance sustaining power through a thin film coated on the skin.

In addition, according to the eye makeup cosmetic composition with the anti-creasing action and the improved sustaining power and the method for preparing the same of the present invention, a functional group including an amide group, a phosphate group or a hydroxyl group is bonded to both ends of the fluorine-based derivative to improve the dispersibility of the cosmetic material and hydrophilic properties are expressed in the functional group, so that the moisturizing property can be sustained upon application to the skin.

In addition, according to the eye makeup cosmetic composition with the anti-creasing action and the improved sustaining power and the method for preparing the same of the present invention, hydrophobic silica ingredients are contained, so that the fluorine-based derivative can be stabilized, and the smooth feeling of use and the clear color can be maintained upon application to the skin.

Further, according to the eye makeup cosmetic composition with the anti-creasing action and the improved sustaining power and the method for preparing the same of the present invention, the eye makeup cosmetic composition enhances the adsorptive power of the fluorine-based derivative by containing the fluorine-based derivative and the hydrophobic silica, and thus can display clear eye makeup while the colors on the skin are not smeared or wiped away for a long period of time.

### DESCRIPTION OF DRAWINGS

FIG. 1 is a flowchart showing a method of preparing an eye makeup cosmetic composition with an anti-creasing action and improved sustaining power according to the present invention.

### BEST MODE

### Mode for Invention

As a result of intensive studies to achieve the above-mentioned technical problems, the present inventors have developed a method of preparing an eye makeup cosmetic composition with an anti-creasing action and improved sustaining power, and have completed the present invention by confirming that the eye makeup cosmetic composition with an anti-creasing action and improved sustaining power, which is prepared by using the above method, prevents the generation of creases by containing a fluorine-based derivative and hydrophobic silica, improves the adhesive property to the skin, enhances sustaining power through a thin film coated on the skin, maintains a smooth feeling of use and clear color when coated on the skin, and displays clear eye makeup while the colors on the skin are riot smeared or wiped away for a long period of time.

The present invention provides an eye makeup cosmetic composition with an anti-creasing action and improved sustaining power, the eye makeup cosmetic composition including a fluorine-based derivative, hydrophobic silica, boron nitride, pearl, extender pigment, vegetable oil, and talc.

The cosmetic composition of the present invention will be described in detail as follows.

The eye makeup cosmetic composition with an anti-creasing action and improved sustaining power according to the present invention includes 5% to 64% by weight of a fluorine-based derivative, 5% to 10% by weight of hydrophobic silica, 10% to 25% by weight of boron nitride, 10% to 20% by weight of pearl, 0.5% to 5% by weight of extender pigment, 0.5% to 5% by weight of vegetable oil, and 10% to 30% by weight of talc.

The fluorine-based derivative is chemically and physically stable, and has excellent stability since ester bond increases stereoscopic degrees of freedom and thus maintains liquid state even at low temperature.

In addition, the fluorine-based derivative has excellent absorbability, so that skin affinity, adhesion and gloss can be remarkable, and film formation and coating power on the skin can be excellent.

Preferably, the fluorine-based derivative may have molecular weight of 500 to 3,000 to use the fluorine-based derivative as a cosmetic material. When the fluorine-based derivative has the molecular weight less than 500, adsorption power of the fluorine-based derivative is decreased, thus the film formation and the gloss on the skin are weakened. When the molecular weight is more than 3,000, the hydrophobic property is enhanced, thus the fluorine-based derivative is rarely properly mixed with cosmetic ingredients, thereby causing poor compatibility and causing a separation, phenomenon between cosmetic ingredients.

The fluorine-based derivative may be polyfluoroalkyl ether, and preferably may be polyperfluoromethylisopropyl ether. The polyperfluoromethylisopropyl ether, which has hydrophobic and lipophilic properties, serves to prevent the color being easily smeared or erased.

The functional group including an amide group, a phosphate group, or a hydroxyl group is bonded to both ends of the fluorine-based derivative, thereby improving the dispersibility of the cosmetic material and expressing hydrophilic properties in the functional group, so that the moisturizing property can be sustained upon application to the skin. Polyperfluoroethoxymethoxy difluoromethyl distearamide is formed when amide group is further bonded to the both ends of the fluorine-based derivative. Polyperfluoroethoxymethoxy difluoroethyl PEG phosphate is formed when phosphate group is further bonded to the both ends of the fluorine-based derivative. Polyperfluoroethoxymethoxy difluorohydroxyethyl ether is formed when hydroxy group is further bonded to the both ends of the fluorine-based derivative.

The fluorine-based derivative is contained in an amount of 5% to 64% by weight based on the total weight of the eye makeup cosmetic composition. When the content of the fluorine-based derivative is less than 5% by weight, durability and soft feeling of use may be lowered, and when the content is more than 64% by weight, the cosmetic material rarely spreads when applied to the skin, and the crease phenomenon cannot be prevented.

The hydrophobic silica stabilizes the fluorine-based derivative and improves the adsorption power of the fluorine-based derivative, thereby serving to display clear eye makeup while the colors around the eyes are not smeared or wiped away for a long period of time. In addition, the hydrophobic silica serves to maintain the form of the formulation, and correct a skin tone and control the moisture around the eyes.

Preferably, the hydrophobic silica includes at least one selected from silica dimethyl silylate, silica dimethicone silylate, silicone acrylate and dimethicone.

The hydrophobic silica is contained in an amount of 5% to 10% by weight based on the total weight of the eye makeup cosmetic composition. When the content of the hydrophobic silica is less than 5% by weight, the fluorine-based derivative is not stabilized, thus the compatibility of the fluorine-based derivative is lowered. When the content is more than 10% by weight, the feeling of use is heavy and stuffy upon application to the skin.

The boron nitride serves to help the fluorine derivative to prevent the crease phenomenon, provides glossiness, soft feeling of use, and reduces the blurring phenomenon around the eyes.

The boron nitride is contained in an amount of 10% to 25% by weight based on the total weight of the eye makeup cosmetic composition. When the content of boron nitride is less than 10% by weight, the glossiness and soft feeling of use are lowered, and when the content is more than 25% by weight, dry feeling of use incurs and the blurring phenomenon cannot be reduced.

The pearl and the extender pigment are used to cover defects of the skin and give a clear color around the eyes.

Preferably, the pearl is formed of at least one selected from titanium dioxide coated on mica, titanium, dioxide coated on borosilicate, and titanium dioxide coated on calcium sodium borosilicate.

Preferably, the extender pigment is selected from at least one of mica, sericite, barium sulfate, alumina, kaolin, calcium carbonate, talc, barium silicate, zeolite, muscovite and magnesium carbonate.

When the content of the pearl is less than 10% by weight and the content of the extender pigment is less than 0.5% by weight based on the total weight of the eye makeup cosmetic composition, the defects of the skin are rarely covered, and the clear color cannot be given around the eyes. When the pearl is more than 20% by weight and the extender pigment is more than 5% by weight, the bonding force between the raw materials increases, thereby causing lumps, and particularly, the dispersing power of the pearl is degraded sharply, thus the pearl is sunk in a product, thereby degrading the stereoscopic feeling upon eye makeup. In addition, the effects on color expression and gloss cannot be obtained with a viewpoint of the feeling of use, because the pearl sinks in a large amount.

The talc serves to help the fluorine-based derivative to prevent the crease phenomenon, improves the adhesion to the skin, and enables the gradation to be effectively formed upon eye makeup. When the content of the talc is less than 10% by weight based on the total weight of the eye makeup cosmetic composition, the adhesion to the skin is degraded, and when the content is more than 30% by weight, the cosmetic material is smeared on an applicator, thus the gradation may be difficult upon makeup.

The vegetable oil serves to manage moisturization, elasticity and exfoliation around the eyes, and preferably includes at least one selected from the group including jojoba seed oil, sunflower oil, grape seed oil, and olive oil.

The vegetable oil is contained in an amount of 0.5% to 5% by weight based on the total weight of the eye makeup cosmetic composition.

When the content of the vegetable oil is less than 0.5% by weight, the moisturizing power around the eyes is lowered, thus the user feels dryness, and when the content is more than 5% by weight, peripheries of the eyes become oily.

Preferably, at least one selected from the group including an antioxidant, an ultraviolet absorbing agent, an ultraviolet screening agent, and a moisturizing agent may be further contained in the eye makeup cosmetic composition with an anti-creasing action and improved sustaining power according to the present invention.

The antioxidant is further contained, such that wrinkles around the eyes can be improved and aging can be prevented. In addition, the ultraviolet absorbing agent and the ultraviolet screening agent are further contained, thereby blocking ultraviolet rays around eyes, so that dermatitis, aging and the like caused by the ultraviolet rays can be prevented. Further, the moisturizing agent is further contained, thereby increasing the moisturizing power around the eyes, such that the dryness around the eyes while moist feeling is given.

The method of preparing an eye makeup cosmetic composition with an anti-creasing action and improved sustaining power according to the present invention will be described with reference to the accompanying drawings.

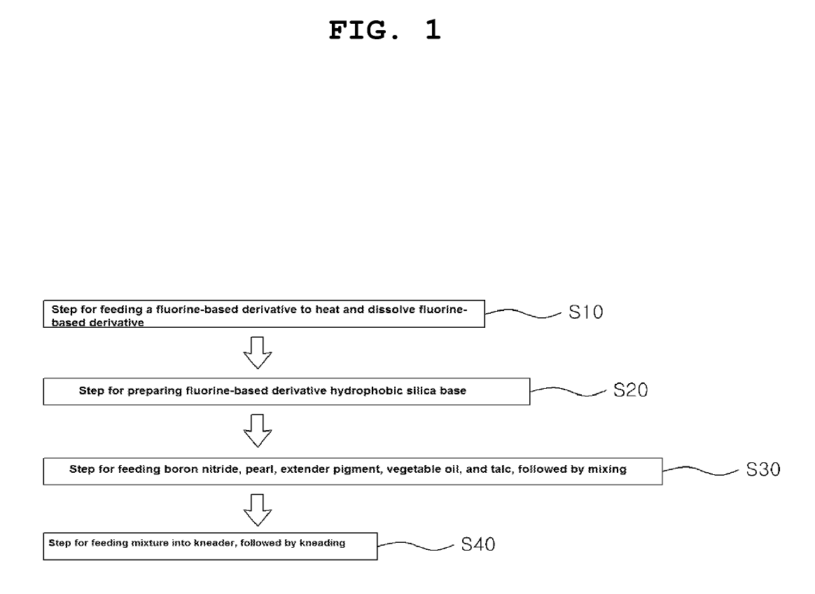

FIG. 1 is a flowchart showing the method of preparing an eye makeup cosmetic composition with an anti-creasing action and improved sustaining power according to the present invention.

Hereinafter, preferable embodiments of the present invention will be described with reference to the accompanying drawings. The method of the present invention preferably includes the following steps, but it is not limited thereto.

The present invention includes the steps of: feeding a fluorine-based derivative into a mixing vessel to heat and dissolve the fluorine-based derivative at 50° C. to 90° C. (S10); feeding hydrophobic silica to the mixing vessel and agitating the mixture by using an agi mixer to prepare a fluorine-based derivative-hydrophobic silica base (S20); sequentially and slowly feeding boron nitride, pearl, extender pigment, vegetable oil and tale to the base to uniformly mix the mixture by using the agi mixer (S30); and feeding the mixed cosmetic material into a kneader to knead the mixed cosmetic material to prepare a final eye makeup cosmetic composition (S40).

The manufacturing method of the present invention will be described separately for each step.

Step S10 is performed in which the fluorine-based derivative is fed to a mixing vessel to heat and dissolve the fluorine-based derivative at 50° C. to 90° C.

The fluorine-based derivative is subjected to a pretreatment step of heating and dissolving at 50° C. to 90° C. to improve the compatibility.

Step S20 is performed in which hydrophobic silica is fed to the mixing vessel and agitated by using an agi mixer to prepare a fluorine-based derivative-hydrophobic silica base.

After step S10, the hydrophobic silica is fed, and the mixture is agitated in the mixing vessel at 500 to 3,000 rpm for 1 to 3 hours, thus the compatibility of the fluorine-based derivative is improved more efficiently, and the stability of the formulation is increased, thereby preparing a fluorine derivative-hydrophobic silica base.

When the agitating speed is less than 500 rpm and the agitating time is less than 1 hour, a portion of the formulation is aggregated. When the agitating speed is more than 3,000 rpm and the agitating time is more than 3 hours, bubbles are generated in the fluorine derivative-hydrophobic silica base, thereby decreasing the viscosity and increasing the defective rate of the eye makeup cosmetic.

Step S30 is performed in which boron nitride, pearl, extender pigment, vegetable oil, and talc are sequentially and slowly fed to the base to uniformly mix the mixture by using the agi mixer.

The pearl and the extender pigment are fed to cover defects of the skin and give a clear color around the eyes. The vegetable oil are fed to provide moisturization and elasticity around the eyes. The boron nitride and the talc serve to help the fluorine-based derivative to prevent the crease phenomenon, and are fed to improve the adhesion to the skin, provide glossiness, soft feeling of use, and reduce a blurring phenomenon around the eyes.

Step S40 is performed in which the mixed cosmetic material is fed and kneaded in a kneader to prepare a final eye makeup cosmetic composition.

In addition, in step S40, at least one selected from the group including an antioxidant, an ultraviolet absorbing agent, an ultraviolet screening agent, and a moisturizing agent may be further contained, such that an eye makeup cosmetic composition with an anti-creasing action and improved sustaining power can be prepared.

Hereinafter, the present invention will be described in detail with examples, comparative examples, and test examples.

The following examples, comparative examples, and test examples are merely illustrative of the present invention and the present invention is not limited to the following examples, comparative examples, and test examples.

### <Examples 1> Example 1 of a Method of Preparing an Eye Makeup Cosmetic Composition with an Anti-Creasing Action and Improved Sustaining Power

Example 1 of a method of preparing an eye makeup cosmetic composition with an anti-creasing action and improved sustaining power according to the present invention was prepared through the following preparing steps by using the contents of the compositions described in Table 1.

S10: A fluorine-based derivative is fed to a mixing vessel to heat and dissolve the fluorine-based derivative at 70° C.

S20: Hydrophobic silica is fed to the mixing vessel and agitated by using an agi mixer at a speed of 2,000 rpm for 1 hour to prepare a fluorine-based derivative-hydrophobic silica base.

S30: Boron nitride, pearl, extender pigment, vegetable oil, and talc are sequentially and slowly fed to the base to uniformly mix the mixture by using the agi mixer.

S40: The mixed cosmetic material is fed and kneaded in a kneader to prepare a final eye makeup cosmetic composition.

### <Comparative Example 1> Comparative Example 1 of a Method of Preparing an Eye Makeup Cosmetic Composition with an Anti-Creasing Action and Improved Sustaining Power

Comparative Example 1 for a method of preparing an eye makeup cosmetic composition with an anti-creasing action and improved sustaining power according to the present invention prepared the composition based on the change of the content of the fluorine-based derivative, and the composition was prepared through the following steps by using the contents of the compositions described in Table 1.

The eye makeup cosmetic composition with an anti-creasing action and Improved sustaining power was prepared in the same manner as Example 1 except that 1% by weight of the fluorine-based derivative was added (less than 5% by weight of the fluorine-based derivative) and the remaining amount was adjusted by the talc.

### <Comparative Example 2> Comparative Example 2 of a Method of Preparing an Eye Makeup Cosmetic Composition with an Anti-Creasing Action and Improved Sustaining Power

Comparative Example 2 for a method of preparing an eye makeup cosmetic composition with an anti-creasing action and improved sustaining power according to the present invention prepared based on the change of the content of the fluorine-based derivative, and the composition was prepared through the following steps by using the contents of the compositions described in Table 1.

The eye makeup cosmetic composition with an anti-creasing action and improved sustaining power was prepared in the same manner as Example 1 except that 70% by weight of the fluorine-based derivative was added (more than 5% by weight of the fluorine-based derivative) and the remaining amount was adjusted by the talc.

### <Comparative Example 3> Comparative Example 3 of a Method of Preparing an Eye Makeup Cosmetic Composition with an Anti-Creasing Action and Improved Sustaining Power

Comparative Example 3 for a method of preparing an eye makeup cosmetic composition with an anti-creasing action and improved sustaining power according to the present invention prepared the composition using dipropylene glycol dibenzoate instead of the fluorine-based derivative, and the composition was prepared through the following preparing steps by using the contents of the compositions described in Table 1.

The eye makeup cosmetic composition with an anti-creasing action and improved sustaining power was prepared in the same manner as Example 1 except that 50% by weight of the dipropylene glycol dibenzoate is added instead of the fluorine-based derivative and the remaining amount is adjusted by the talc.

TABLE 1
Unit: wt %

| | | Embodiment | Comparative | Comparative | Comparative |
|---|---|---|---|---|---|
| Item | Ingredients | 1 | Example 1 | Example 2 | Example 3 |
| | Dipropylene Glycol | X | X | X | 50 |
| | Dibenzoate | | | | |
| fluorine- | polyperfluoromethyl- | 50 | 1 | 70 | X |
| based | isopropyl ether | | | | |
| derivative | | | | | |
| hydrophobic | silica dimethyl | 2.5 | 2.5 | 2.5 | 2.5 |
| silica | silylate | | | | |
| | silica dimethicone | 2 | 2 | 2 | 2 |
| | silylate | | | | |
| | silicone acrylate | 2 | 2 | 2 | 2 |
| | dimethicone | 1 | 1 | 1 | 1 |
| boron | boron nitride | 17.5 | 17.5 | 17.5 | 17.5 |
| nitride | | | | | |
| pearl | calcium sodium | 15 | 15 | 15 | 15 |
| | borosilicate + | | | | |
| | titanium dioxide | | | | |
| extender | zeolite, maca | 2.75 | 2.75 | 2.75 | 2.75 |
| pigment | | | | | |
| vegetable | sunflower oil | 1.5 | 1.5 | 1.5 | 1.5 |
| oil | grape seed oil | 1.25 | 1.25 | 1.25 | 1.25 |
| talc | talc | to 100 | to 100 | to 100 | to 100 |

### <Test Example> Evaluation on an Eye Makeup Cosmetic Composition with an Anti-Creasing Action and Improved Sustaining Power According to the Content of a Fluorine-Based Derivative and the Presence or Absence of the Fluorine-Based Derivative

With respect to the compositions of Example: 1 and Comparative Examples 1 to 3, the results of use were compared and evaluated after allowing fifty females in their 20 s and 30 s to use the eye makeup cosmetic composition with art anti-creasing action and improved sustaining power for 7 to 10 days.

The sensory evaluation was performed in feeling of use, blurring (erasing), uniformity of application, inhibition of crease, stability, and sustainability. The evaluation criteria were indicated by 1 (very unsatisfactory) to 10 (very satisfied). The results are shown in Table 2.

TABLE 2

| | | | uni- | inhibi- | | |
|---|---|---|---|---|---|---|
| | | | formity | tion | | |
| | | | of | of | | |
| | | | appli- | crease | | |
| | feeling | | cation | (degree | | |
| | of use | | (close | of | | |
| | (spread- | | contact | crease on | | |
| | ability, | blurring/ | with | double | | sustain- |
| Item | touch) | erasing | skin) | eyelid) | stability | ability |
| Embodiment | 9.8 | 9.7 | 9.8 | 9.8 | 9.5 | 9.7 |
| 1 | | | | | | |
| Comparative | 7.3 | 7.1 | 7.4 | 7.2 | 7.3 | 7.1 |
| Example 1 | | | | | | |
| Comparative | 6.8 | 7.1 | 7.2 | 6.9 | 7.3 | 7.4 |
| Example 2 | | | | | | |
| Comparative | 6.7 | 6.4 | 6.4 | 5.9 | 6.1 | 6.2 |
| Example 3 | | | | | | |

As shown in Table 2, Example 1 and Comparative Examples 1 and 2 were evaluated according to the content of fluorine-based derivative, and Example 1 and Comparative Example 3 were evaluated according, to the presence or absence of the fluorine-based derivative.

Comparative Example 1 contains less than 5% by weight of fluorine-based derivative (contains 1% by weight of fluorine-based derivative), in which it was found that the compatibility with other cosmetic ingredients was decreased because the hydrophobic property of the fluorine-based derivative was not mitigated, and as a result, the shape of the formulation was unstable.

In addition, the film was not properly formed around the eyes, thus the skin adhesion was reduced, and the cosmetic material was creased between the double eyelid.

In addition, the appearance seemed dull because of lowered glossiness and the durability was lowered.

Comparative Example 2 contained more than 64% by weight of fluorine-based derivative (contains 70% by weight of fluorine-based derivative), the separation phenomenon between cosmetic ingredients incurred.

In addition, because the compatibility of the fluorine-based derivative was not improved, the crease restraining power was lower than that of Comparative Example 1, thus the cosmetic material were creased in double eyelid or around the eyes.

In addition, it was found that the part around the eyes seemed dirty because the color was blurred upon application to the skin, and the feeling of use was heavy and stuffy because the film was formed thicker around the eye than in Example 1.

In Comparative Example 3, dipropylene glycol dibenzoate was used instead of the fluorine-based derivative, it was difficult to form a thin film around the eyes, and stickiness incurred and that the part around the eyes was oily upon application to the skin.

In addition, the stereoscopic effect was reduced upon eye makeup, and the effects on the color expression and gloss could not be obtained with a viewpoint of the feeling of use because the pearl sank in a large amount. The creased phenomenon incurred in which the cosmetic material for eye makeup was creased upon eye makeup. In 1 to 2 hours after completion of the eye makeup, the makeup was blurred or wiped away, and the double eyelid line was separated from the cosmetic material for eye makeup, thus the part around the eyes seemed to be dirty and to have wrinkles.

However, it was confirmed that Example 1 prevents the generation of the crease, improves the adhesive property to the skin, enhances sustaining power through a thin film coated on the skin, maintains a smooth feeling of use and clear color when coated on the skin, and displays clear eye makeup while the colors on the skin was not smeared or wiped away for a long period of time.

Accordingly, it was found that Example 1 was superior to Comparative Examples 1 to 3 in the feeling of use, blurring (erasing), uniformity of application, inhibition of crease, stability, and sustainability, and it was confirmed that the fluorine-based derivative should be present within a certain amount range so as to obtain the anti-creasing action and the sustaining power.

Herein, the description of the present invention is merely an embodiment for the eye makeup cosmetic composition with an anti-creasing action and improved sustaining power and the method for preparing the same. The present invention is not limited to the above embodiments, and it may be implemented in various different forms. The embodiments are provided to complete the disclosure of the present invention, and to clearly teach the scope of the invention to those skilled in the art of the present invention. The present invention is only defined by the scope of the claims.

1. An eye makeup cosmetic composition with an anti-creasing action and improved sustaining power, the eye makeup cosmetic composition comprising a fluorine-based derivative, hydrophobic silica, boron nitride, pearl, extender pigment, vegetable oil, and talc.

2. An eye makeup cosmetic composition with an anti-creasing action and improved sustaining power, the eye makeup cosmetic composition comprising 5% to 64% by weight of a fluorine-based derivative, 5% to 10% by weight of hydrophobic silica, 10% to 25% by weight of boron nitride, 10% to 20% by weight of pearl, 0.5% to 5% by weight of extender pigment, 0.5% to 5% by weight of vegetable oil, and 10% to 30% by weight of talc.

3. The eye makeup cosmetic composition of claim 1, wherein the fluorine-based derivative has a molecular weight of 500 to 3,000.

4. The eye makeup cosmetic composition of claim 1, wherein the fluorine-based derivative includes polyperfluoromethylisopropyl ether.

5. The eye makeup cosmetic composition of claim 1, or wherein a functional group including an amide group, a phosphate group or a hydroxyl group is bonded to both ends of the fluorine-based derivative.

6. The eye makeup cosmetic composition of claim 1, wherein the hydrophobic silica includes at least one selected from silica dimethyl silylate, silica dimethicone silylate, silicone acrylate and dimethicone.

7. A method of preparing an eye makeup cosmetic composition with an anti-creasing action and improved sustaining power, the method comprising:
feeding a fluorine-based derivative into a mixing vessel to heat and dissolve the fluorine-based derivative at 50° C. to 90° C. (S10);
feeding hydrophobic silica to the mixing vessel and agitating the mixture by using an agi mixer to prepare a fluorine-based derivative-hydrophobic silica base (S20);
sequentially and slowly feeding boron, nitride, pearl, extender pigment, vegetable oil and talc to the base and uniformly mixing the mixture by using the agi mixer (S30); and
feeding the mixed cosmetic material to a kneader to knead the mixed cosmetic material.

8. The method of claim 7, wherein, in S20, the agitating is performed at 500 to 3,000 rpm for 1 to 3 hours.

9. The eye makeup cosmetic composition of claim 2, wherein the fluorine-based derivative has a molecular weight of 500 to 3,000.

10. The eye makeup cosmetic composition of claim 2, wherein the fluorine-based derivative includes polyperfluoromethylisopropyl ether.

11. The eye makeup cosmetic composition of claim 2, wherein a functional group including an amide group, a phosphate group or a hydroxyl group is bonded to both ends of the fluorine-based derivative.

12. The eye makeup cosmetic composition of claim 2, wherein the hydrophobic silica includes at least one selected from silica dimethyl silylate, silica dimethicone silylate, silicone acrylate and dimethicone.
